(19) United States
(12) Patent Application Publication
(10) Pub. No.: US 2011/0312894 A1
(43) Pub. Date: Dec. 22, 2011

(54) METHODS OF DIAGNOSING AND TREATING NEURODEGENERATIVE DISEASES

(75) Inventor: Jie Wu, Avondale, AZ (US)

(73) Assignee: CATHOLIC HEALTHCARE WEST, Phoenix, AZ (US)

(21) Appl. No.: 13/146,625

(22) PCT Filed: Jan. 28, 2010
(86) PCT No.: PCT/US10/22424

§ 371 Date: Jul. 27, 2011

Related U.S. Application Data
(60) Provisional application No. 61/148,010, filed on Jan. 28, 2009.

(51) Int. Cl. A61K 38/02; G01N 21/76; A61K 31/47; A61P 25/28; A61K 31/55; A61K 31/13; A61P 25/08; G01N 33/566; C12Q 1/68

(52) U.S. Cl. 514/17.7; 435/7.21; 436/501; 514/312; 435/6.11; 514/216; 514/662

(57) ABSTRACT

The present invention relates to methods of diagnosing, treating and prognosing mental disorders, such as Alzheimer's Disease. In one embodiment, the present invention provides a method of treating Alzheimer's Disease by inhibiting dysfunctional signaling of α7 nAChRs in the medial septum region of an individual.

### GOVERNMENT RIGHTS

This invention was made with government support under Contract No. ROI DA015389 awarded by the National Institutes of Health. The government has certain rights in the invention.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Nicotinic acetylcholine receptors (nAChRs) in mammals exist as a diverse family of channels composed of different, pentameric combinations of subunits derived from at least sixteen genes (Lukas et al., 1999; Jensen et al., 2005). Functional nAChRs can be assembled as either heteromers containing [symbol] and β subunits or as homomers containing only [symbol] subunits (Lukas et al., 1999; Jensen et al., 2005). In the mammalian brain, the most abundant forms of nAChRs are heteromeric α4β2-nAChRs and homomeric α7-nAChRs (Whiting et al., 1987; Flores et al., 1992; Gopalakrishnan et al., 1996; Lindstrom, 1996; Lindstrom et al., 1996). α7-nAChRs appear to play roles in the development, differentiation, and pathophysiology of the nervous system (Liu et al., 2007b; Mudo et al., 2007).

nAChRs have been implicated in Alzheimer's disease (AD), in part because significant losses in radioligand binding sites corresponding to nAChRs have been consistently observed at autopsy in a number of neocortical areas and in the hippocampi of patients with AD (Burghaus et al., 2000; Nordberg, 2001). Attenuation of cholinergic signaling is known to impair memory, and nicotine exposure improves cognitive function in AD patients (Levin and Rezvani, 2002). In addition, several studies have suggested that the activation of α7-nAChR function alleviates amyloid-β (Aβ) toxicity. For instance, stimulation of α7-nAChRs inhibits amyloid plaque formation in vitro and in vivo (Geerts, 2005), activates α-secretase cleavage of amyloid precursor protein (APP) (Lahiri et al., 2002), increases acetylcholine (ACh) release and facilitates Aβ internalization (Nagele et al., 2002), inhibits activity of the MAPKINF-kB/c-myc signaling pathway (Liu et al., 2007a), and reduces Aβ production and attenuates tau phosphorylation (Sadot et al., 1996). These findings suggest that cholinergic signaling, mediated through α7-nAChRs, not only is involved in cognitive function, but also could protect against a wide variety of insults associated with AD (Sivaprakasam, 2006). Conversely, impairment of α7-nAChR-mediated cholinergic signaling during the early stage(s) of AD might play a pivotal role in AD pathophysiology.

In rat basal forebrain cholinergic neurons, α7 and β2 are the predominant nAChR subunits, and they were found to co-localize (Azam et al., 2003). Thus far, there has been no evidence that α7 and β2 subunits co-assemble to form functional nAChRs naturally, although functional α7β2-nAChRs have been reported using a heterologous expression system (Khiroug et al., 2002). As described herein, however, the inventors demonstrate that heteromeric α7β2-nAChRs exist in rodent basal forebrain cholinergic neurons and have high sensitivity to Aβ. There is a need in the art for a greater understanding of the role of nAChRs in learning and memory disorders, specifically Alzheimer's Disease, both in their functional characterization as well as the development of novel treatments for Alzheimer's Disease.

### SUMMARY OF THE INVENTION

Various embodiments include a method of treating a neurodegenerative disorder in an individual, comprising providing a composition capable of inhibiting dysfunctional signaling of α7 nicotinic acetylcholine receptors (nAChRs), and administering a therapeutically effective amount of the composition to inhibit dysfunctional signaling of α7 nAChRs to treat the neurodegenerative disorder. In another method, the α7 nAChRs comprise heteromeric α7β2 nAChRs. In another embodiment, the composition comprises a β2 nAChR antagonist. In another embodiment, the neurodegenerative disorder comprises Alzheimer's Disease, dementia and/or epilepsy. In another embodiment, the neurodegenerative disorder comprises an early stage form of Alzheimer's Disease. In another embodiment, the composition comprises an α7 nAChR antagonist. In another embodiment, the composition comprises a therapeutically effective amount of compound comprising kynurenic acid (KYNA), methyllycaconitine (MLA), α-bungarotoxin (BGT), cholinesterase inhibitor, memantine, and/or α-conotoxin, or a pharmaceutical equivalent, derivative, analog and/or salt thereof. In another embodiment, inhibiting the dysfunctional signaling of α7 nAChRs comprises restoring function of heteromeric α7β2 nAChRs. In another embodiment, inhibiting the dysfunctional signaling of α7 nAChRs comprises protecting heteromeric α7β2 nAChRs from amyloid β (Aβ) effects. In another embodiment, the individual is a human. In another embodiment, the individual is a rodent. In another embodiment, the dysfunctional signaling of α7 nAChRs occurs in the brain medial septum and/or diagonal band in the individual.

Other embodiments include a method of diagnosing a neurodegenerative disorder in an individual, comprising obtaining a sample from the individual, assaying the sample to determine the presence or absence of dysfunctional signaling of α7 nicotinic acetylcholine receptors (nAChRs) in the individual, and diagnosing the neurodegenerative disorder based on the presence of dysfunctional signaling of α7 nAChRs in the individual. In another embodiment, the α7 nAChRs comprise heteromeric α7β2 nAChRs. In another embodiment, the individual is a human. In another embodiment, the individual is a rodent. In another embodiment, the neurodegenerative disorder comprises Alzheimer's Disease, dementia and/or epilepsy. In another embodiment, the dysfunctional signaling of α7 nAChRs occurs in the brain medial septum and/or diagonal band in the individual. In another embodiment, the neurodegenerative disorder has proven non responsive to treatment with galantamine, or a pharmaceutical equivalent, derivative, analog and/or salt thereof. In another embodiment, prior to obtaining the sample the individual is suspected of having a neurodegenerative disorder.

Various embodiments include a method of prognosing the onset of Alzheimer's Disease and/or dementia in an individual, comprising obtaining a sample from the individual, assaying the sample to determine the presence or absence of dysfunctional signaling of α7 nicotinic acetylcholine receptors (nAChRs) in the individual, and prognosing the onset of Alzheimer's Disease and/or dementia based on the presence of dysfunctional signaling of α7 nAChRs in the individual. In another embodiment, the α7 nAChRs comprise heteromeric α7β2 nAChRs. In another embodiment, the dysfunctional signaling of α7 nAChRs occurs in the brain medial septum and/or diagonal band in the individual.

Other embodiments include a method of diagnosing an increased likelihood of developing a neurodegenerative disorder relative to a normal subject in an individual, comprising obtaining a sample from the individual, assaying the sample to determine the presence or absence of dysfunctional signaling of α7 nicotinic acetylcholine receptors (nAChRs) in the individual, and diagnosing an increased likelihood of developing the neurodegenerative disorder relative to a normal subject based on the presence of dysfunctional signaling of α7 nAChRs in the individual. In another embodiment, the α7 nAChRs comprise heteromeric α7β2 nAChRs. In another embodiment, the neurodegenerative disorder comprises Alzheimer's Disease, dementia and/or epilepsy. In another embodiment, prior to obtaining the sample the individual is suspected of having a neurodegenerative disorder.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the identification of cholinergic neurons dissociated from basal forebrain. A: Phase contrast image of a rat MS/DB brain slice (region confirmed using The Rat Brain in Stereotaxic Coordinates, Paxinos and Watson, 1986). MS/DB neurons (phase-contrast images of dissociated neurons; B) exhibited spontaneous action potential firing (C), insensitivity to muscarine (C), action potential adaptation induced by depolarizing pulses (D), and did not show ‘sag’-like responses to hyperpolarizing pulses (E), suggesting they were cholinergic. F: Dissociated neuron (phase contrast, Ph) labeled with lucifer yellow (LY) showed positive ChAT immunostaining following patch-clamp recording.

FIG. 2 depicts native nAChR-mediated whole-cell current responses. An identified MS/DB cholinergic neuron (no hyperpolarization-induced current, Iₕ) exhibited α7-nAChR-like current responses to 1 mM ACh and 10 mM choline (sensitive to blockade by 1 nM methyllycaconitine; MLA) but not to 0.1 mM RJR-2403, an agonist selective for α4β2-nAChRs (A), whereas an identified VTA DAergic neuron (evident Iₕ) showed both α7-nAChR-like (i.e., choline and MLA-sensitive components) and α4β2-nAChR-like (i.e., RJR-2403-sensitive component) current responses (summed as in the response to ACh) (B). C: typical traces of 10 mM choline-induced currents in MS/DB and VTA DAergic neurons showing different kinetics for current activation/desensitization with a slower response characteristic of MS/DB neurons. D: statistical comparisons of kinetics of 10 mM choline-induced currents in MS/DB cholinergic and VTA DAergic neurons. ***p<0.001.

FIG. 3 depicts nAChR α7 and β2 subunits are co-expressed, co-localize and co-assemble in rat forebrain MS/DB neurons. RT-PCR products from whole brain, VTA and MS/DB regions (A) corresponding to the indicated nAChR subunits or to the housekeeping gene GAPDH were resolved on an agarose gel calibrated by the flanking 100 bp ladders (heavy band is 500 bp) and visualized using ethidium staining. Note that the representative gel shown for whole brain did not contain a sample for the nAChR α3 subunit RT-PCR product, which typically is similar in intensity to the sample on the gel for the VTA and MS/DB. B: quantification of nAChR subunit mRNA levels for RT-PCR amplification followed by Southern hybridization with ³²P-labeled, nested oligonucleotides normalized to the GAPDH internal control and to levels of each specific mRNA in whole rat brain (ordinate: ±S.E.M.) for the indicated subunits. C: From 15 MS/DB neurons tested, after patch-clamp recordings (Ca: representative whole-cell current trace) the cell content was harvested and single-cell RT-PCR was performed, and the results show that α7 and β2 were the two major nAChR subunits naturally expressed in MS/DB cholinergic neurons (Cb-Cd). Double immunofluorescence labeling of a MS/DB neuron using anti-α7 and anti-β2 subunit antibodies revealed that α7 and β2 subunit proteins co-localized, and similar results were obtained using 31 neurons from 12 rats (D). Protein extracts from rat MS/DB (lane 1) or rat VTA (lane 2) or from MS/DB from nAChR β2 subunit knockout (lane 4) or wild-type mice (lane 5) were immunoprecipitated (IP) with a rabbit anti-α7 antibody (Santa Cruz H302; lanes 1, 2, 4, and 5) or rabbit IgG as a control (lane 3). The eluted proteins from the precipitates were analyzed by immunoblotting (IB) with rat monoclonal anti-β2 subunit antibody mAb270 (upper panel) or rabbit anti-α7 antisera H302 (lower panel). The β2 and α7 bands are indicated by arrows (E). All these data demonstrate that nAChR α7 and β2 nAChR subunits are co-assembled in MS/DB neurons.

FIG. 4 depicts antagonist profiles for MS/DB and VTA nAChRs. Concentration-dependent block by MLA (at the indicated concentrations in nM after pre-exposure for 2 min and continued exposure during agonist application indicated by open bars) of 10 mM choline-induced (applied as indicated by closed bars) whole-cell currents (representative traces shown) in MS/DB (Aa) and VTA (Ab) neurons was not significantly different (p>0.05, Ac). However, choline-induced currents in MS/DB neurons (Ba) were more sensitive to block by DHβE (at the indicated concentrations μM after pre-exposure for 2 min and continued exposure during agonist application indicated by open bars) than in VTA neurons (Bb; concentration-response profile shown in Bc).

FIG. 5 depicts effects of 1 nM Aβ₁₋₄₂ on α7β2-nAChRs on MS/DB neurons. Typical whole-cell current traces for responses of MS/DB neurons to 10 mM choline challenge at the indicated times after initial challenge alone show no detectable rundown during repetitive application of agonist (2-s exposure at 2-min intervals; Aa). Choline-induced currents in rat MS/DB neurons were suppressed by 1 nM Aβ₁₋₄₂ (continuously applied for 10 min, but responses to challenges with choline are shown at the indicated times of Aβ exposure; Ab) but not by 1 nM scrambled Aβ₁₋₄₂ (as a control; Ac). Choline-induced currents in VTA neurons were not affected by 1 nM Aβ₁₋₄₂ (Ad). B: Normalized, mean (±SE), peak current responses (ordinate) as a function of time (abscissa, min) during challenges with choline alone (□), in the presence of 1 nM Aβ (▴), or in the presence of control, scrambled Aβ (▾) for the indicated numbers of MS/DB neurons, or during challenges with choline in the presence of 1 nM Aβ for the indicated number of VTA neurons () illustrate that only choline-induced currents in rat MS/DB neurons were sensitive to functional inhibition by Aβ.

FIG. 6 depicts inhibition of choline-induced currents in dissociated MS/DB neurons by Aβ₁₋₄₂ was concentration- and form-dependent. A: Normalized, mean (±SE), peak current responses (ordinate) of the indicated numbers of MS/DN neurons as a function of time (abscissa, min) during challenges with choline in the presence of 1 nM scrambled Aβ (▪) or in the presence of 0.1 nM (), 1 nM (▴) or 10 nM (▾) Aβ show concentration dependence of functional block. B: Normalized responses (ordinate) during challenges with choline in the presence of 1 nM monomeric (▪), oligomeric (▴) or fibrillar () Aβ indicate insensitivity to monomeric Aβ and highest sensitivity to peptide oligomers. *p<0.05, **p<0.01, and ***p<0.001.

FIG. 7 depicts effects of Aβ on heterologously-expressed, homomeric α7- and heteromeric α7β2-nAChRs in Xenopus oocytes. Choline (10 mM, 2-s exposure at 2-min intervals)-induced whole-cell current responses in oocytes injected with rat α7-nAChR subunit tRNA alone (Aa, black trace) or with α7 and β2 subunit cRNAs at a ratio of 1:1 (Aa) show slower decay of elicited currents and a longer decay time constant for heteromeric receptors (Aa and b). The scale bars represent 1 sec and 1 μA for the α7-nAChR response (black trace) and 1 sec and 100 nA for the α7β2-nAChR response, thus also showing that current amplitudes were lower for heteromeric than for homomeric receptors. B: Normalized, mean (±SE), peak current responses (ordinate) of the indicated numbers of oocytes heterologously expressing nAChR α7 and β2 subunits (▪, ) or only α7 subunits (▴) as a function of time (abscissa, min) during challenges with choline alone (▪) or in the presence of 1.0 nM Aβ (, ▴) show sensitivity to functional block by Aβ only for heteromeric receptors. *p<0.05, **p<0.01, and ***p<0.001.

FIG. 8 depicts kinetics, pharmacology and Aβ sensitivity of α7-containing-nAChRs in nAChR β2 subunit knockout mice. Genotype analyses demonstrated that nAChR β2 subunits are not expressed in nAChR β2 knockout mice (A), whereas Lac-Z (as a marker for the knockout) was absent in wild-type (WT) mice (B). Kinetic analyses showed that whole-cell current kinetics and amplitudes differed for MS/DB neurons from WT compared to nAChR β2 subunit knockout homozygote mice (Ca,b). Compared to MS/DB neurons from WT mice (Da), choline-induced currents in MS/DB (Db) neurons from β2 knockouts were insensitive to DHβE but retained sensitivity to MLA (Dc). 1 nM Aβ₁₋₄₂ suppressed choline-induced currents in MS/DB neurons from WT (▪) but not from β2 knockout () mice (E). ‘Control’ responses (▴) were choline-induced currents in neurons from WT mice without exposure to Aβ₁₋₄₂. *p<0.05, **p<0.01.

FIG. 9 depicts atomic force microscopic (AFM) images of different forms of Aβ1-42. A: Images and height distribution analysis of Aβ1-42 at 0, 2 and 4 h following stock solution preparation showing time-dependent increase in Aβ aggregation. C: Aβ1-42 (diluted to 100 nM as stock solutions) was prepared using different protocols to obtain AFM imaging-confirmed, monomeric, oligomeric or fibrillar forms.

FIG. 10 depicts effects of 1 nM Aβ1-42 on ligand-gated ion channel activity in rat MS/DB neurons. A: typical whole-cell current response traces (left-to-right) before, after 6 or 10 min of exposure to 1 nM Aβ1-42, or after washout of peptide on 0.1 mM GABA- (a), 1 mM glutamate- (Glu, b), or 1 mM ACh- (c) induced currents. B. Mean (±SEM) normalized peak current responses (ordinate) as a function of time (abscissa, min; Aβ exposure from 0-10 min) from 4-12 neurons to 1 mM ACh (), 1 mM glutamate (Glu; ▴) or 0.1 mM GABA (▪). *p<0.05, **p<0.01.

FIG. 11 depicts pharmacological profiles for nAChR antagonist action at heterologously expressed α7- or α7β2-nAChRs in oocytes. Concentration-dependent block by MLA (at the indicated concentrations in nM after pre-exposure for 2 min indicated by open bars) of 10 mM choline-induced (applied as indicated by closed bars) whole-cell currents (representative traces shown) elicited in oocytes injected with nAChR α7 and β2 subunit cRNA (A) or only with α7 subunit cRNA (B) was not significantly different (p>0.05, n=5, C). However, choline-induced currents in oocytes expressing α7β2-nAChRs (D) were more sensitive (F) to block by DHβE (at the indicated concentrations in μM after pre-exposure for 2 min and continued exposure during agonist application indicated by open bars) than currents mediated by homomeric α7-nAChRs (E).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, the term. “Aβ” refers to amyloid beta peptides.

As used herein, the term “nAChR” refers to nicotinic acetylcholine receptor.

As used herein, the term “Aβ₁₋₄₂” refers to amyloid beta peptides at positions 1-42 of the amyloid precursor protein (APP).

As used herein, the term “MS/DB” means medial septum/diagonal band.

As used herein, the term “AD” means Alzheimer's Disease.

As used herein, the term “dysfunctional signaling” refers to signaling mechanisms that are considered to be abnormal and not ordinarily found in a healthy subject or typically found in a population examined as a whole with an average amount of incidence.

As used herein, “treatment” or “treating” should be understood to include any indicia of success in the treatment, alleviation or amelioration of an injury, pathology or condition. This may include parameters such as abatement, remission, diminishing of symptoms, slowing in the rate of degeneration or decline, making the final point of degeneration less debilitating; improving a patient's physical or mental well-being; or, in some situations, preventing the onset of disease.

As used herein, “diagnose” or “diagnosis” refers to determining the nature or the identity of a condition or disease. A diagnosis may be accompanied by a determination as to the severity of the disease.

As used herein, “prognostic” or “prognosis” refers to predicting the outcome or prognosis of a disease.

As disclosed herein, nicotinic acetylcholine receptors (nAChRs) containing α7 subunits are believed to assemble as homomers. α7-nAChR function has been implicated in learning and memory, and alterations of α7-nAChR have been found in patients with Alzheimer's disease (AD). Findings in rodent, basal forebrain holinergic neurons are described herein consistent with a novel, naturally occurring nAChR subtype. In these cells, α7 subunits are coexpressed, colocalize, and coassemble with β2 subunit(s). Compared with homomeric α7-nAChRs from ventral tegmental area neurons, functional, heteromeric α7β2-nAChRs on cholinergic neurons freshly dissociated from medial septum/diagonal band (MS/DB) exhibit relatively slow kinetics of whole-cell current responses to nicotinic agonists and are more sensitive to the β2 subunit-containing nAChR-selective antagonist, dihydro-β-erythroidine (DH βE). Interestingly, heteromeric α7β2-nAChRs are highly sensitive to functional inhibition by pathologically relevant concentrations of oligomeric, but not monomeric or fibrillar, forms of amyloid β₁₋₄₂ (Aβ₁₋₄₂). Slow whole-cell current kinetics, sensitivity to DHβE, and specific antagonism by oligomeric Aβ₁₋₄₂ also are characteristics of heteromeric α7β2-nAChRs, but not of homomeric α7-nAChRs, heterologously expressed in Xenopus oocytes. Moreover, choline-induced currents have faster kinetics and less sensitivity to Aβ when elicited from MS/DB neurons derived from nAChR β2 subunit knock-out mice rather than from wild-type mice. The presence of novel, functional, heteromeric α7β2-nAChRs on basal forebrain cholinergic neurons and their high sensitivity to blockade by low concentrations of oligomeric Aβ₁₋₄₂ supports the existence of mechanisms for deficits in cholinergic signaling that could occur early in the etiopathogenesis of AD and could be targeted by disease therapies.

In one embodiment, the present invention provides a method of diagnosing susceptibility to a learning and/or memory disorder by determining the presence or absence of dysfunctional signaling of α7 containing nAChRs in a subject, where the presence of dysfunctional signaling of α7 containing nAChRs is indicative of susceptibility to the learning and/or memory disorder. In another embodiment, the α7 containing nAChRs comprise heteromeric α7β2-nAChRs. In another embodiment, the learning and/or memory disorder is Alzheimer's Disease. In another embodiment, the α7 containing nAChRs are found in basal forebrain cholinergic neurons. In another embodiment, the subject is a rodent. In another embodiment, the subject is a human.

In another embodiment, the present invention provides a method of diagnosing a learning and/or memory disorder by determining the presence or absence of dysfunctional signaling of α7 containing nAChRs in a subject, where the presence of dysfunctional signaling of α7 containing nAChRs is indicative of the learning and/or memory disorder. In another embodiment, the α7 containing nAChRs comprise heteromeric α7β2-nAChRs. In another embodiment, the learning and/or memory disorder is Alzheimer's Disease. In another embodiment, the α7 containing nAChRs are found in basal forebrain cholinergic neurons. In another embodiment, the subject is a rodent. In another embodiment, the subject is a human.

In one embodiment, the present invention provides a method of treating a learning and/or memory disorder in a subject by determining the presence of dysfunctional signaling of α7 containing nAChRs and inhibiting the dysfunctional signaling of α7 containing nAChRs. In another embodiment, the learning and/or memory disorder is Alzheimer's Disease. In another embodiment, inhibiting dysfunctional signaling of α7 containing nAChRs includes inhibiting expression of the nAChR α7 subunit. In another embodiment, inhibiting heteromeric α7β2-nAChR dysfunctional signaling includes the inhibition of expression of the nAChR β2 subunit. In another embodiment, the inhibition of expression of the nAChR β2 subunit includes fast whole-cell kinetics and/or low sensitivity to amyloid beta peptides.

As readily apparent to one of skill in the art, any number of readily available materials and known methods may be used to inhibit or activate nAChR signaling. For example, α7 nAChR antagonists such as α-conotoxin analogs (Armishaw, et al, Journal of Biological Chemistry, Vol. 285, No. 3; Armishaw, et al., Journal of Biological Chemistry, Vol. 284 No. 14), memantine (Aracava, et al., Journal of Pharmacology and Experimental Therapeutics, Vol. 312, No. 3), and kynurenic acid (Hilmas, et al., Journal of Neuroscience, 21(19): 7463-7473), may be used in conjunction with various embodiments herein to inhibit signaling of α7 containing nAChRs.

In various embodiments, the present invention provides pharmaceutical compositions including a pharmaceutically acceptable excipient along with a therapeutically effective amount of compound that results in the inhibition of dysfunctional signaling of nAChRs. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, on as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Typical dosages of an effective composition that results in the inhibition of dysfunctional signaling of nAChRs can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generally

Nicotinic acetylcholine receptors (nAChRs) containing α7 subunits are believed to assemble as homomers. α7-nAChR function has been implicated in learning and memory, and alterations of α7-nAChR have been found in patients with Alzheimer's disease (AD). Findings in rodent, basal forebrain holinergic neurons are described herein consistent with a novel, naturally occurring nAChR subtype. In these cells, α7 subunits are coexpressed, colocalize, and coassemble with β2 subunit(s). Compared with homomeric α7-nAChRs from ventral tegmental area neurons, functional, heteromeric α7β2-nAChRs on cholinergic neurons freshly dissociated from medial septum/diagonal band (MS/DB) exhibit relatively slow kinetics of whole-cell current responses to nicotinic agonists and are more sensitive to the β2 subunit-containing nAChR-selective antagonist, dihydro-P-erythroidine (DH βE). Interestingly, heteromeric α7β2-nAChRs are highly sensitive to functional inhibition by pathologically relevant concentrations of oligomeric, but not monomeric or fibrillar, forms of amyloid β₁₋₄₂ (Aβ₁₋₄₂). Slow whole-cell current kinetics, sensitivity to DHβE, and specific antagonism by oligornericAβ₁₋₄₂ also are characteristics of heteromeric α7β2-nAChRs, but not of homomeric α7-nAChRs, heterologously expressed in Xenopus oocytes. Moreover, choline-induced currents have faster kinetics and less sensitivity to Aβ when elicited from MS/DB neurons derived from nAChR β2 subunit knock-out mice rather than from wild-type mice. The presence of novel, functional, heteromeric α7β2-nAChRs on basal forebrain cholinergic neurons and their high sensitivity to blockade by low concentrations of oligomeric Aβ₁₋₄₂ supports the existence of mechanisms for deficits in cholinergic signaling that could occur early in the etiopathogenesis of AD and could be targeted by disease therapies.

### Example 2

### Acutely-Dissociated Neurons from the CNS and Patch-Clamp Whole-Cell Current Recordings

Neuron dissociation and patch clamp recordings were performed as described in (Wu et al., 2002; Wu et al., 2004b). Briefly, each postnatal 2-4 week-old Wistar rat or mouse (wild-type C57/B16 or nAChR β2 knockout mice on a C57/B16 background kindly provided by Dr. Marina Picciotto, Yale University) was anesthetized using isoflurane, and the brain was rapidly removed. Several 400-μm coronal slices, which contained the medial septum/diagonal band (MS/DB) or the ventral tegmental area (VTA), were cut using a vibratome (Vibratorne 1000 plus; Jed Pella Inc., Redding, Calif.) in cold (2-4° C.) artificial cerebrospinal fluid (ACSF) and continuously bubbled with carbogen (95% O₂-5% CO₂). The slices were then incubated in a pre-incubation chamber (Warner Ins., Holliston, Mass.) and allowed to recover for at least 1 h at room temperature (22±PC) in oxygenated ACSF. Thereafter, the slices were treated with pronase (1 mg/6 mL) at 31° C. for 30 min and subsequently treated with the same concentration of thermolysin for another 30 min. The MS/DB or VTA region was micropunched out from the slices using a well-polished needle. Each punched piece was then dissociated mechanically using several fire-polished micro-Pasteur pipettes in a 35-mm culture dish filled with well-oxygenated, standard external solution (in mM: 150NaCl, 5KCl, 1MgCl₂, 2CaCl₂, 10 glucose 10, and 10 HEPES; pH 7.4 (with Tris-base). The separated single cells usually adhered to the bottom of the dish within 30 min. Perforated-patch whole-cell recordings coupled with a U-tube or two-barrel drug application system were employed (Wu et al., 2002). Perforated-patch recordings closely maintain both intracellular divalent cation and cytosolic element composition (Horn and Marty, 1988). In particular, perforated-patch recording was used to maintain the intracellular ATP concentration at a physiological level. To prepare for perforated-patch whole-cell recording, glass microelectrodes (GC-1.5; Narishige, East Meadow, N.Y.) were fashioned on a two-stage vertical pipette puller (P-830; Narishige, East Meadow, N.Y.), and the resistance of the electrode was 3 to 5 MΩ when filled with the internal solution. A tight seal (>2 GΩ) was formed between the electrode tip and the cell surface, which was followed by a transition from on-cell to whole-cell recording mode due to the partitioning of amphotericin B into the membrane underlying the patch. After whole-cell formation, an access resistance lower than 60 MΩ was acceptable during perforated-patch recordings in current-clamp mode, and an access resistance lower than 30 MΩ was acceptable during voltage-clamp recordings. The series resistance was not compensated in the experiments using dissociated neurons. Under current-clamp configuration, membrane potentials were measured using a patch-clamp amplifier (200B; Axon Instruments, Foster City, Calif.). Data was filtered at 2 kHz, acquired at 11 kHz, and digitized on-line (Digidata 1322 series A/D board; Axon Instruments, Foster City, Calif.). All experiments were performed at room temperature (22±1° C.). The drugs used in the present study were GABA, glutamate, ACh, choline, methyllycaconitine (MLA), dihydro-β-erythroidine (DHβE), muscarine (all purchased from Sigma-Aldrich, St. Louis, Mo.), RJR-2403 (purchased from Tocris Cookson Inc., Ballwin, Mo.), and Aβ₁₋₄₂ and scrambled Aβ₁₋₄₂ (purchased from rPeptide, Athens, Ga.).

### Example 3

### RT-PCR to Profile nAChR Subunit Expression in MS/DR

Riboprobe construction: Templates for in vitro transcription were created using PCR and sense or antisense primers spanning the 5′ SP6 promoter or the 3′ T7 promotor, respectively (α7 subunit: 5′-atttaggtgacaetatagaagnggatcatcgtgggcctetcagtg-3′ (SEQ. 1D. NO.: 1) and 5′-taatacgactcactatagggagagaggcgatgtageggacctc-3′ (SEQ. ID. NO.: 2); β2 subunit: 5′-atttaggtgacactatagaagngtcacggtgttectgctgctcatct-3′(SEQ. ID. NO.: 3) and 5′-taatacgactcactatagggagatcctccetcacactctggtcatca-3′ (SEQ. ID. NO.: 4)). Antisense or sense probes were then created by in vitro transcription using SP6 or T7 polymerases, respectively, and by incorporation of biotin-tagged UTP (for β2 subunit probes) or digoxigenin-tagged UTP (for α7 subunit probes; biotin or digoxigenin RNA labeling mix; Roche Applied Science, Indianapolis, Ind.). 433 bp or 520 bp products corresponded to mRNA nucleotides 953-1385 for α7 subunits or mRNA nucleotides 1006-1525 for β2 subunits thus produced are highly specific to the individual subunits.

Tissue RT-PCR: RT-PCR assays followed by Southern hybridization with nested oligonucleotides were done as previously described to identify nAChR subunit transcripts and to quantify levels of expression normalized both to housekeeping gene expression and levels of expression in whole brain (Zhao et al., 2003; Wu et al., 2004), but using primers designed to detect rat nAChR subunits. The Southern hybridization technique coupled with quantitation using electronic isotope counting (Instant Imager, Canaberra Instruments, Meridien, Conn.) yielded results equivalent to those obtained using real-time PCR analysis.

Single-cell RT-PCR: Precautions were taken to ensure a ribonuclease-free environment and to avoid PCR product contamination during patch-clamp recording and single-cell collection prior to execution of RT-PCR. Single-cell RT-PCR was performed using the Superscript III CellDirect RT-PCR system (Invitrogen, Carlsbad, Calif.). Briefly, after whole-cell patch-clamp recording, single-cell content was harvested by suction into the pipette solution (˜3 μL) and immediately transferred to an autoclaved 0.2 mL PCR tube containing 10 μ [symbol] of cell resuspension buffer and 1 μL of lysis enhancer. Single cells were lysed by heating at 75° C. for 10 min. Potential contaminating genomic DNA was removed by DNase I digestion at 25° C. for 6 min. After heat-inactivation of DNaseI at 70° C. for 6 min in the presence of EDTA, reverse transcription (RT) was performed by adding reaction mix with oligo(dT)₂O and random hexamers and SuperSciptIII enzyme mix and then incubating at 25° C. for 10 min and 50° C. for 50 min. The reaction was terminated by heating the sample to 85° C. for 5 min. The PCR primers for glyceraldehyde-3-phosphate dehydrogenase (GAPDH) and nAChR α3, α4, α7, β2 and β4 subunits were designed using the Primer 3 internet server (MIT) and assuming an annealing temperature of ˜60° C. [nearest neighbor]. PCR was performed with 20 μ [symbol] of hot-start Platinum PCR Supermix (Invitrogen, Carlsbad, Calif.), 3 μ [symbol] of cDNA template from the RT step, and 1 μ [symbol] of gene specific primer pairs (5 pmole each) with the following thermocycling parameters: 95° C. for 2 mM; (95° C. for 30 s, 60° C. for 30 s, and 72° C. for 40 s)×70 cycles, 72° C. for 1 min. PCR products were resolved on 1.5% TBE-agarose gels, and stained gels were used to visualize bands, employing digital photography and a gel documentation system to capture images.

### Example 4

### Tissue Protein Extraction, Immunoprecipitation, and Immunoblotting for Confirmation of nAChR α7 and β2 Subunit Co-Assembly

Tissues were Dounce homogenized (10 strokes) in ice-cold lysis buffer (1% (v/v) Triton X-100, 150 mM EDTA, 10% (v/v) glycerol, 50 mM Tris-HCl, pH 8.0) containing 1× general protease inhibitor cocktails (Sigma-Aldrich, St. Louis, Mo.). The lysates were transferred to microcentrifuge tubes and further solubilized for 30 min at 4° C. The detergent extracts (supernatants) were collected by centrifugation at 15,000 g for 15 min at 4° C., and protein concentration was determined for sample aliquots using bicinchoninic acid (BCA) protein assay reagents (Pierce Chemical Co., Rockford, Ill.). The detergent extracts were then precleared with 50 μL of mixed slurry of protein A-Sepharose and protein G-Sepharose (1:1) (Amersham Biosciences, Piscataway, N.J.) twice, each for 30 min at 4° C. For each immunoprecipitation, detergent extracts (1 mg) were mixed with 1 μg of rabbit anti-α7 antisera (H302) or rabbit IgG (as immunological control) (Santa Cruz Biotechnology, Inc., Santa Cruz, Calif.) and incubated at 4° C. overnight with continuous agitation. Protein A-Sepharose and protein G-Sepharose mixtures (50 μL) were added and incubated at 4° C. for 1 h. The beads were washed four times with ice-cold lysis buffer containing protease inhibitors. Laemmli sample buffer eluates were resolved by SDS-PAGE. Proteins were transferred onto Hybond ECL nitrocellular membranes (Amershan Biosciences, Sunnyvale, Calif.). The membranes were blocked with TBST buffer (20 mM Tris-HCl (pH 7.6), 150 mM NaCl, and 0.1% (v/v) Tween 20) containing 2% (w/v) non-fat dry milk for at least 2 h and incubated with rat monoclonal anti-β2 antibody (mAb270; Santa Cruz, Calif.) or anti-α7 antisera (H302), respectively, at 4° C. overnight. After three washes in TBST, the membranes were incubated with goat anti-rat or goat anti-rabbit secondary antibodies (1:10,000) (Pierce Chemical Co., Rockford, Ill.) for 1 h and washed. The bound antibodies were detected with SuperSignal chemiluminescent substrate (Pierce Chemical Co., Rockford, Ill.).

### Example 5

### Expression of Homomeric and Heteromeric α7-Containing-nAChRs in Xenopus oocytes and Two-Electrode Voltage-Clamp Recording

cDNAs encoding rat α7 and β2 subunits were amplified by PCR with pfuUltra DNA polymerase and subcloned into an oocyte expression vector, pGEMHE, with T7 orientation and confirmed by automated sequencing. cRNAs were synthesized by standard in vitro transcription with T7 RNA polymerase, confirmed by electrophoresis for their integrity, and quantified based on optical absorbance measurements using an Eppendorf Biophotometer.

Oocyte preparation and cRNA injection: Female Xenopus laevis (Xenopus I, Ann Arbor, Mich.) were anesthetized using 0.2% MS-222. The ovarian lobes were surgically removed from the frogs and placed in an incubation solution consisting of (in mM): 82.5NaCl, 2.5KCl, 1MgCl₂, 1CaCl₂, 1Na₂HPO₄, 0.6 theophylline, 2.5 sodium pyruvate, 5 HEPES, 50 mg/mL gentamycin, 50 U/mL penicillin and 50 μg/mL streptomycin; pH 7.5. The frogs were then allowed to recover from surgery before being returned to the incubation tank. The lobes were cut into small pieces and digested with 0.08 Wunsch U/mL liberase blendzyme 3 (Roche Applied Science, Indianapolis, Ind.) with constant stirring at room temperature for 1.5-2 h. The dispersed oocytes were thoroughly rinsed with incubation solution. Stage VI oocytes were selected and incubated at 16° C. before injection. Micropipettes used for injection were pulled from borosilicate glass (Drummond Scientific, Broomall, Pa.). cRNAs encoding α7 or β2 at proper dilution were injected into oocytes separately or in different ratios using a Nanoject microinjection system (Drummond Scientific, Broomall, Pa.) at a total volume of ˜20-60 nL.

Two-electrode voltage-clamp recording: One to three days after injection, an oocyte was placed in a small-volume chamber and continuously perfused with oocyte Ringer's solution (OR2), consisting of (in mM): 92.5NaCl, 2.5KCl, 1CaCl₂, 1MgCl₇ and 5 HEPES; pH 7.5. The chamber was grounded through an agarose bridge. The oocytes were voltage-clamped at −70 mV to measure ACh (or choline)-induced currents using GeneClamp 500B (Axon Instruments, Foster City, Calif.).

### Example 6

### Immunocytochemical Staining

Dissociated MS/DB neurons were fixed with 4% paraformaldehyde for 5 min, rinsed three times with PBS, and treated with saponin (1 mg/mL) for 5 min as a permeabilizing agent. After rinsing four times with PBS, the neurons were incubated at room temperature in anti-choline acetyltransferase (ChAT) primary antibody (AB305; Chemicon International, Temecula, Calif.) diluted 1:400 in Hank's balanced salt solution (supplemented with 5% bovine serum albumin as a blocking agent) for 30 min. Following another three rinses with PBS, a secondary antibody (anti-mouse IgG; Sigma-Aldrich) was applied at room temperature for 30 min (diluted 1:100). After rinsing a final three times with PBS, the labeled cells were visualized using a Zeiss fluorescence microscope (Zeiss, Oberkochen, Germany), and images were processed using Photoshop (Adobe Systems Inc., San Jose, Calif.). For double immunolabeling of α7 and β2 subunits of nAChRs on single dissociated MS/DB neurons, the following antibodies were used: a rabbit antibody (AS-5631S, 1:400; R and D, Las Vegas, Nev.) against α7 subunit, a rat antibody against β2 subunit (Ab24698, 1:500; Abeam, Cambridge, Mass.), Alexa Fluor 594-conjugated anti-rabbit IgG, and Alexa Fluor 488-conjugated anti-rat IgG; (1:300; Molecular Probes, Calif.).

### Example 7

### Aβ Preparation and Determination/Monitoring of Peptide Forms

Aβ preparation: Amyloid β peptides (Aβ₁₋₄₂) were purchased from rPeptide Corn (Athens, Ga.). As previously described (Wu et al., 2004a), some preparations involved reconstitution of Aβ peptides per vendor specifications in distilled water to a concentration of 100 μM, stored at −20° C., and used within 10 days of reconstitution. These thawed peptide stock solutions were used to create working dilutions (1-100 nM) in standard external solution before patch-clamp recording. Working dilutions were used within 4 hours before being discarded. Atomic force microscopy (AFM) was employed to define and analyze over time the morphology of prepared Aβ₁₋₄₂. Aliquots of freshly prepared samples of Aβ₁₋₄₂ diluted in standard external solution were spotted on freshly cleaved mica. After 2 min the mica was washed with 200 μL of deionized water, dried with compressed nitrogen, and completely air-dried under vacuum. Images were acquired in air using a multimode AFM nanoscope IIIA system (Veeco/Digital Instruments, Plainview, N.Y.) operating in the tapping mode using silicon probes (Olympus, Center Valley, Pa.).

Protocols to obtain different forms of Aβ₁₋₄₂: Different conditions were utilized to specifically prepare monomeric, oligomeric or fibrillar forms of Aβ₁₋₄₂.

Monomers: Aβ₁₋₄₂ was reconstituted in DMSO to a concentration of 100 μM and stored at −80° C. For each use, an aliquot of stock sample was freshly thawed and diluted into standard extracellular solution as above just before patch recordings and used for no more than 4 h. This protocol yielded a predominant, monomeric form.

Oligomers: Aβ₁₋₄₂ reconstituted in distilled water to a concentration of 100 μM and stored at −80° C. was used within 7 d of reconstitution. Aliquots diluted in standard extracellular solution and used within 4 h yielded a predominantly oligomeric form.

Fibrils: Aliquots of Aβ₁₋₄₂ stock solution (water dissolved to 100 μM) were thawed and incubated at 37° C. for 48 h at low pH (pH=6.0). Working stocks diluted in standard extracellular solution yielded a predominantly fibrillar form.

### Example 8

### Genotyping of the nAChR β2 Subunit Knockout Mice

Genomic DNA from mice newly born to heterozygotic, nAChR β2 subunit knockout parents was extracted from mouse tail tips using the QIAgen DNeasy Blood & Tissue Kit following the manufacture's protocol. PCR amplification of the nAChR β2 subunit or lac-Z (an indicator for the knockout) were performed using the purified genomic DNA as template and gene specific primer pairs (forward primer: CGG AGC ATT TGA ACT CTG AGC AGT GGG GTC GC (SEQ. ID. NO.: 5); backward primer: CTC GCT GAC ACA AGG GCT GCG GAC (SEQ. ID. NO.: 6); lac-Z forward primer: CAC TAC GTC TGA ACG TCG AAA ACC CG (SEQ. ID. NO.: 7); backward primer: CGG GCA AAT AAT ATC GGT GGC CGT GG (SEQ. ID. NO.: 8)) with annealing at 55° C. for 1 min and extension at 72° C. for 1 min for 30 cycles with GO Taq DNA polymerase (Promega, Madison, Wis.). PCR products were resolved on 1% agarose gels and stained for visualization before images were captured using digital photography.

### Example 9

### Identification of Cholinergic Neurons Dissociated from Basal Forebrain

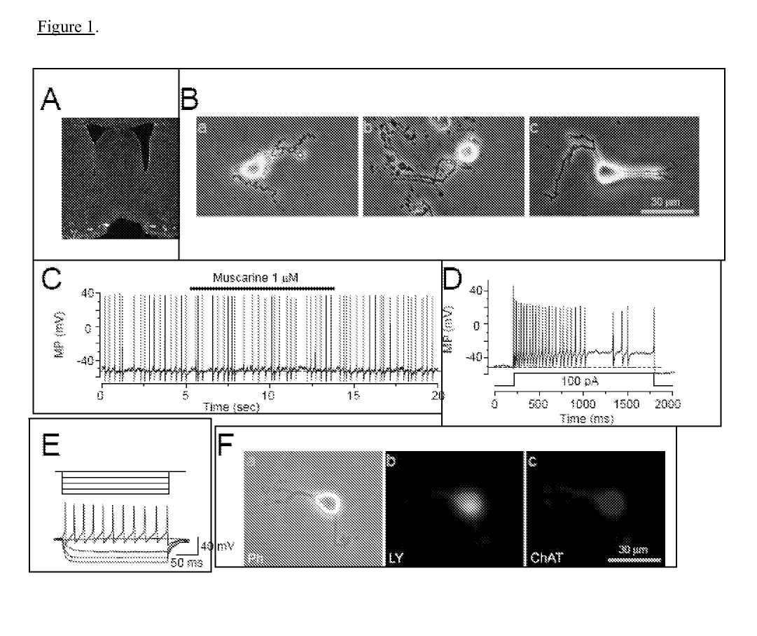

An initial series of experiments identified cholinergic neurons acutely dissociated from rat MS/DB (FIG. 1A). First, the cholinergic phenotype of acutely-dissociated neurons were identified from the MS/DB (FIG. 1Ba-c) based on published criteria (Henderson et al., 2005; Thinschmidt et al., 2005). In current-clamp mode, MS/DB neurons exhibited spontaneous action potential firing at low frequency (2.3±0.4 Hz, n=25 from 21 rats). This spontaneous activity was insensitive to the muscarinic acetylcholine receptor agonist, muscarine (1 μM) (FIG. 1C). Depolarizing pulses induced adaptation of action potential firing (FIG. 1D), and hyperpolarizing pulses failed to induce ‘sag’-like membrane potential changes (FIG. 1E). In some cases, the fluorescent dye lucifer yellow (0.5 mg/mL) was delivered into recorded cells after patch-clamp recordings, and choline acetyltransferase (ChAT) immunocytostaining was employed post-hoc (FIG. 1F). The presence of ChAT immunoactivity in recorded, dye-filled neurons confirmed that dissociated MS/DB neurons were cholinergic.

### Example 10

### Naturally-Occurring nAChRs in Rodent Forebrain Cholinergic Neurons

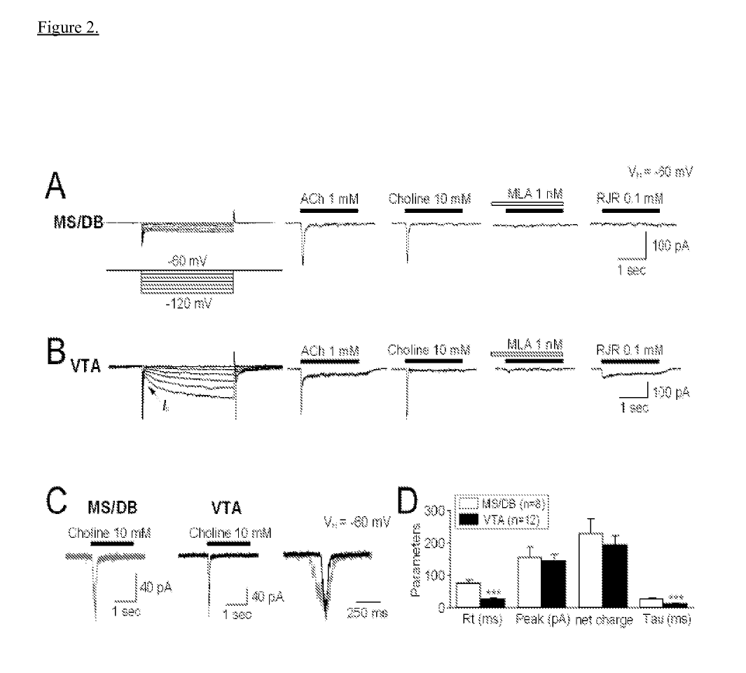

The inventors next tested for the presence of functional nAChRs on MS/DB cholinergic neurons. Under voltage-clamp recording conditions, rapid application of 1 mM ACh induced inward current responses with relatively rapid activation and desensitization kinetics (FIG. 2A). These ACh-induced responses were mimicked by application of the selective α7-nAChR agonist choline, blocked by the relatively-selective α7-nAChR antagonist methyllycaconitine (MLA), and insensitive to the relatively-selective α4β2-nAChR agonist RJR-2403 (FIG. 2A). Thus, the inward current evoked in MS/DB neurons had features similar to receptors containing α7 subunits. By contrast, in acutely-dissociated, dopaminergic (DAergic) neurons from the midbrain VTA, ACh-induced currents displayed a mixture of features that could be dissected pharmacologically and with regard to whole-cell current kinetics. Components of responses displaying slow kinetics and sustained, steady-state currents elicited by ACh were mimicked by RJR-2403, demonstrating that they were mediated by α4β2-nAChRs, whereas choline only induced transient peak current responses with very fast kinetics that are characteristic of homomeric α7-nAChRs (FIG. 2B). Interestingly, choline-induced currents in MS/DB cholinergic neurons exhibited relatively slow macroscopic kinetics than observed in VTA DAergic neurons (FIG. 2C). This impression was confirmed by quantitative analyses, which gave values for current rising time of 72.1±9.1 ms (n=8) for MS/DB neurons and 29.1±2.9 ins (n=12) for VTA neurons (p<0.001) and decay constants (tau, rate of decay from peak to steady state current) of 28.6±2.8 ms (n=8) for MS/DB neurons and 10.2±1.5 ms (n=12) for VTA neurons (p<0.001). There were no significant differences between either peak current amplitudes or net charge movements for responses elicited by choline in MS/DB or VTA neurons (FIG. 2D). These results demonstrated that functional nAChRs naturally expressed on rat MS/DB cholinergic neurons with some features like α7-nAChRs had slower whole-cell current kinetics than found for α7-nAChR-like responses in VTA DAergic neurons.

### Example 11

### Subunit Partnership for Naturally-Occurring nAChRs in Rodent Basal Forebrain Cholinergic Neurons

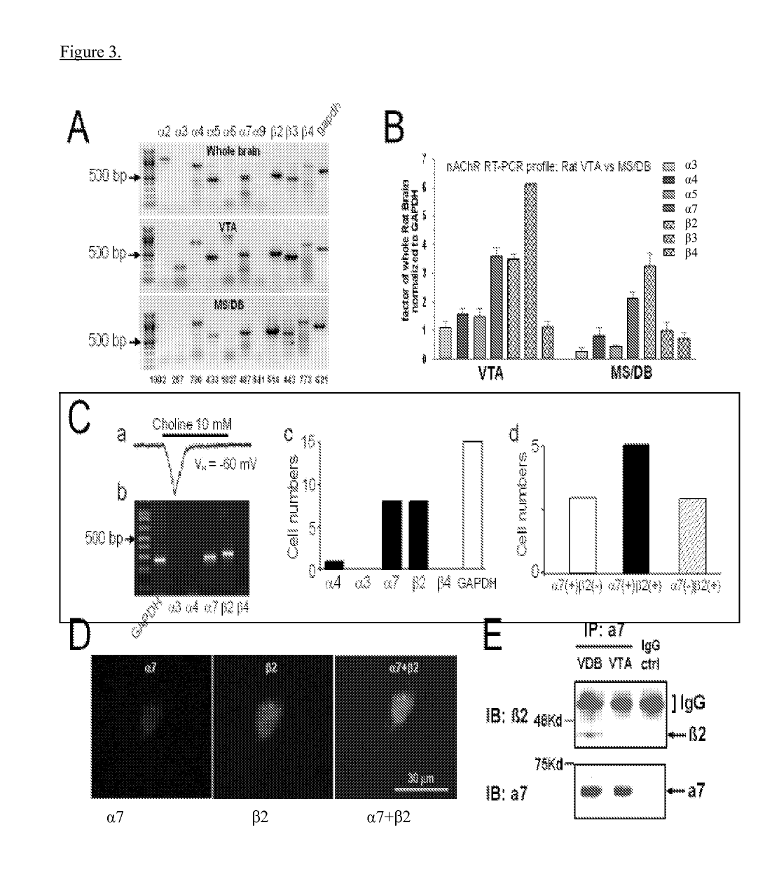

With regard to relatively slow kinetics of α7-nAChR-like responses in MS/DB cholinergic neurons due to co-assembly of α7 with other nAChR subunits, the inventors performed relative quantitative RT-PCR analysis of nAChR subunit expression as messenger RNA in MS/DB compared to whole-brain and VTA tissues. The results demonstrated that nAChR α7 and β2 subunits were among those co-expressed regionally (FIG. 3A, B). These studies were extended to single-cell RT-PCR analysis of nAChR subunit expression in acutely-dissociated neurons from the MS/DB used in patch-clamp recordings (FIG. 3Ca-c). Quantitative analysis indicated a high frequency of nAChR α7 and β2 subunit co-expression as message in recorded MS/DB neurons (FIG. 3Cd). Mindful of the current concerns about the specificity of all anti-nAChR subunit antibodies (Moser et al., 2007), nevertheless it was shown qualitatively, based on dual-labeling immunofluorescent staining (FIG. 3D), that α7 and β2 subunits were co-localized in many MS/DB neurons subjected to patch-clamp recording. More direct evidence for co-assembly of nAChR α7 and β2 subunit proteins came from co-immunoprecipitation studies using subunit-specific antibodies. Protein extracts from rat MS/DB or VTA tissues (collected from rats aged between 18-22 days) were subjected to immunoprecipitation (IP; FIG. 3E; left panel) with a rabbit anti-nAChR α7 subunit antibody (H302) or with rabbit IgG (as an immunological control) followed by immunoblotting (IB) with a rat anti-nAChR β2 subunit monoclonal antibody (mAb270). As indicated herein, the β2 subunit was readily detected immunologically in anti-α7 immunoprecipitates from MS/DB but not from VTA regions under our experimental conditions (FIG. 3E, upper left panel, lane 1 vs. 2). Reprobing the same blot with the rabbit anti-α7 antibody (H302) verified that similar amounts of α7 subunits were precipitated from both MS/DB and VTA regions (FIG. 3E, lower left panel, lanes 1 and 2). Thus, co-precipitation of nAChR α7 and β2 subunits appeared only in samples from the rat MS/DB but not from the VTA. Collectively, these results demonstrate that nAChR α7 and β2 subunits are most likely co-assembled, perhaps to form a functional nAChR subtype, in rodent basal forebrain cholinergic neurons.

### Example 12

### Pharmacological Profiles of Functional nAChRs in Rat Forebrain Cholinergic Neurons

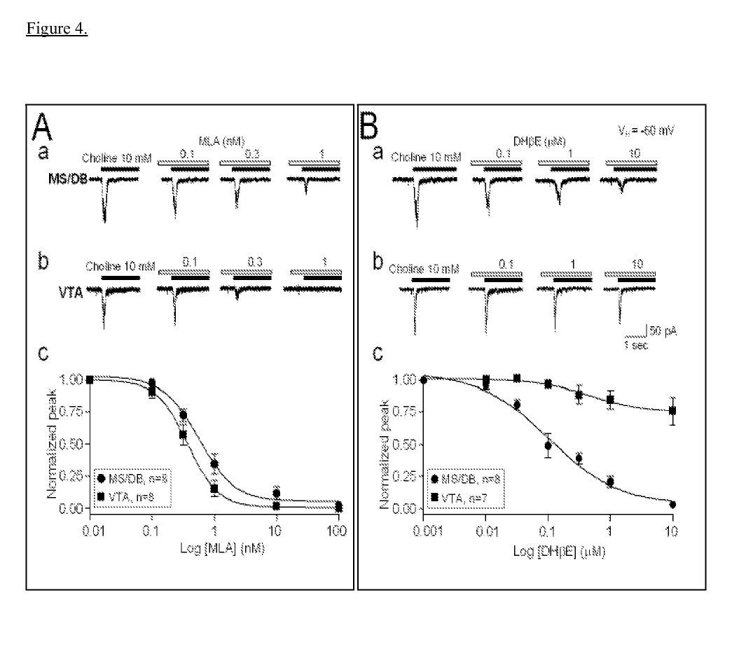

Pharmacological approaches were used to compare features of functional nAChRs in MS/DB cholinergic or VTA DAergic neurons. The α7-nAChR-selective antagonist, MLA showed similar antagonist potency toward choline-induced currents in either MS/DB (FIG. 4Aa) or VTA (FIG. 4Ab) neurons. Analysis of concentration-inhibition curves (FIG. 4Ac) yielded IC₅₀ values and Hill coefficients of 0.7 nM and 1.1, respectively, for MS/DB neurons (n=8) and 0.4 nM and 1.2, respectively, for VTA neurons (n=9, MS/DB vs. VTA p>0.05). However, the β2*-nAChR-selective antagonist, DHβE was ˜500-fold less potent as an inhibitor of choline-induced current in MS/BD neurons (FIG. 4Ba) than in VTA neurons (FIG. 48b). IC₅₀ values and Hill coefficients for DHβE-induced inhibition were 0.17 μM and 0.9, respectively, for MS/DB neurons (n=8), and >100 μM and 0.3, respectively, for VTA neurons (n=7; MS/DB vs. VTA, p<0.001; FIG. 4Bc). These results are consistent with the concept that functional α7*-nAChRs on MS/DB cholinergic neurons also contain DHβE-sensitive β2 subunits.

### Example 13

### Functional nAChRs on Rat Basal Forebrain Cholinergic Neurons are Inhibited by Aβ₁₋₄₂

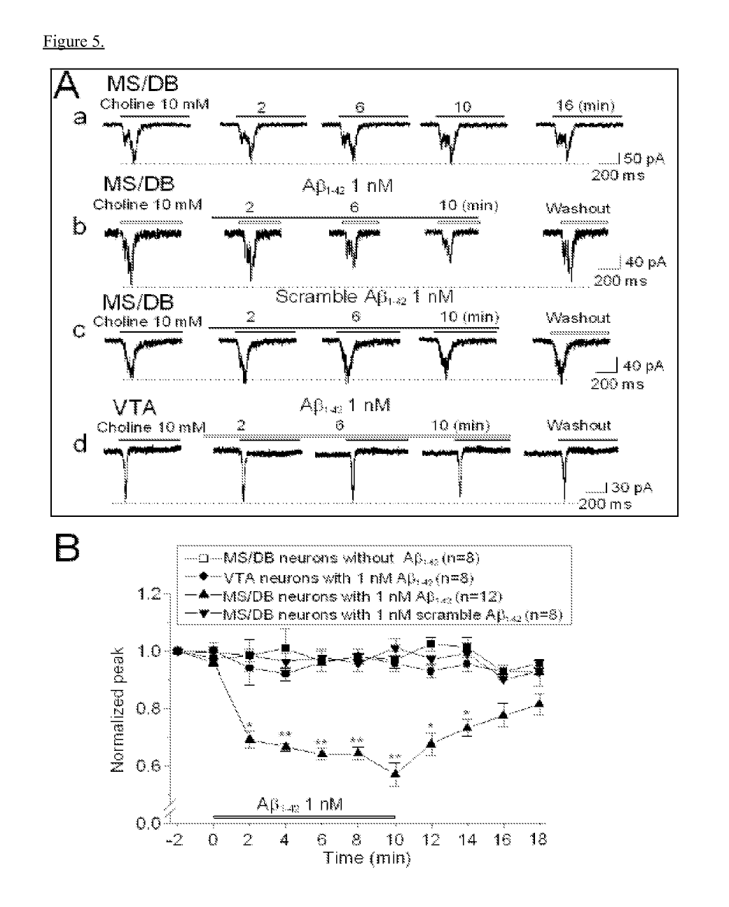

Basal forebrain cholinergic neurons are particularly sensitive to degeneration in AD. To demonstrate that novel α7β2-nAChRs on MS/DB cholinergic neurons are involved, the inventors determined the effects of Aβ₁₋₄₂ on these receptors. The experimental protocol involved repeated, acute challenges with 10 mM choline, and control studies in the absence of peptide demonstrated that there was no significant rundown of such responses when spaced at a minimum of 2-min intervals (FIG. 5Aa). During a continuous exposure to 1 nM Aβ₁₋₄₂ starting just after an initial choline challenge and continuing for 10 min, responses to choline challenges were progressively inhibited with time, although reversibly so as demonstrated by response recovery after 6 min of peptide washout (FIG. 5Ab). By contrast, exposure to 1 nM scrambled Aβ₁₋₄₂ (as a control peptide) had no effect (FIG. 5Ac). Choline-induced currents in dissociated VTA DAergic neurons were not sensitive to 1 nM Aβ₁₋₄₂ treatment (FIG. 5Ad). Quantitative analysis of several replicate experiments (FIG. 5B) confirmed that Aβ₁₋₄₂, even at 1 nM concentration, specifically inhibits putative α7β2-nAChR function on MS/DB cholinergic neurons but not function of homomeric α7-nAChRs on VTA DAergic neurons.

### Example 14

### Concentration- and Form-Dependent Inhibition by Aβ₁₋₄₂ of α7β2-nAChR Function on Basal Forebrain Cholinergic Neurons

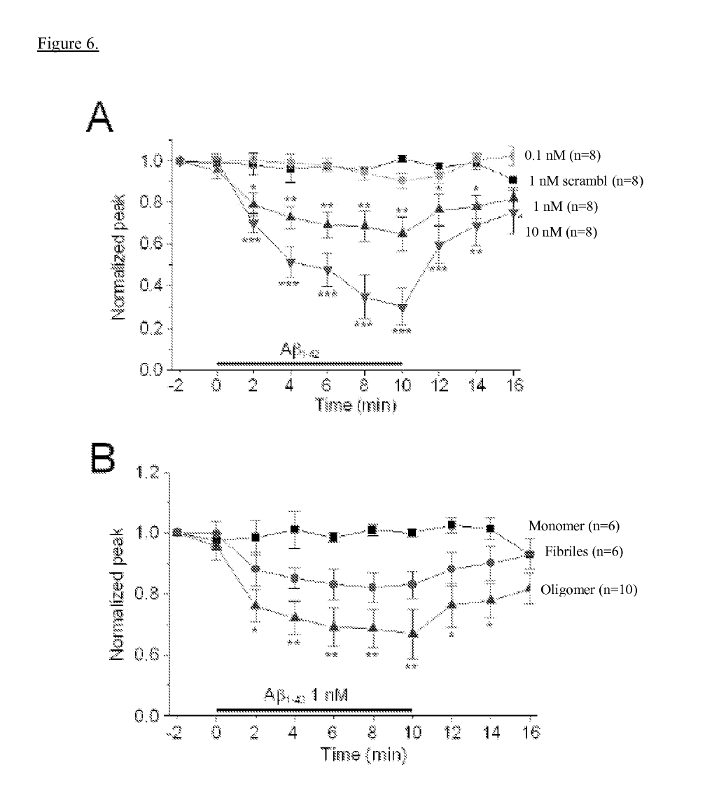

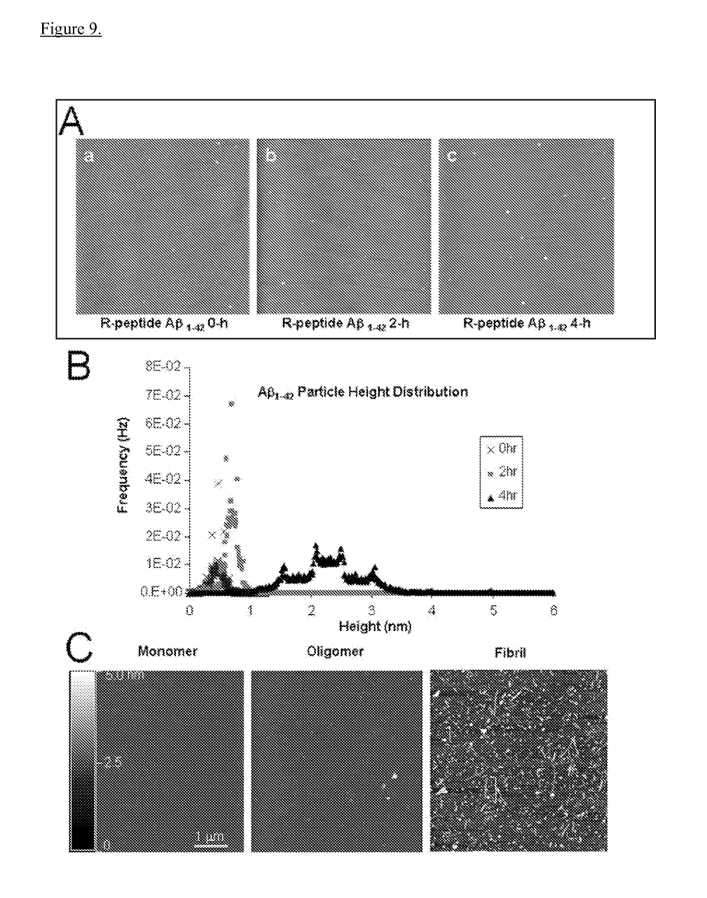

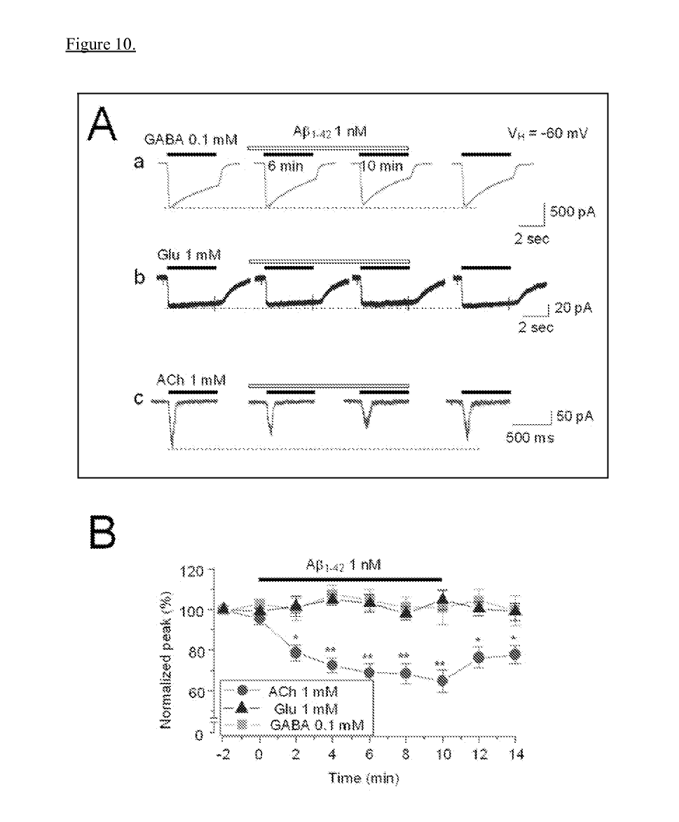

The inventors' previous studies indicated that α4β2-nAChRs were more sensitive to Aβ₁₋₄₂ than homomeric α7-nAChRs (Wu et al., 2004a). Concentration dependence of effects of Aβ₁₋₄₂ on choline-induced currents in MS/DB neurons was evident, with effects being negligible at 0.1 nM and effects at 1 nM being about half of those observed for 10 nM peptide (FIG. 6A). The magnitude of inhibition apparently had not yet reached maximum after 10 min of peptide exposure. The inventors also determined which form(s) of Aβ₁₋₄₂ showed the most potent inhibitory effect on choline-induced currents elicited in MS/DB neurons. Using different preparation protocols, the inventors produced Aβ₁₋₄₂ monomers (peptide dissolved in DMSO), oligomers (peptide dissolved in water), or fibrils (peptides dissolved in water at low pH (pH=6.0) and incubated at 37° C. for 2 days). Peptide forms were defined and monitored using AFM (see FIG. 9). At 1 nM, oligomeric Aβ₁₋₄₂ exhibited the greatest suppression of choline-induced responses, fibrillar Aβ had weaker inhibitory effect, and monomeric Aβ₁₋₄₂ failed to suppress choline-induced responses, indicating form-selective, Aβ₁₋₄₂ inhibition of nAChRs in MS/DB cholinergic neurons. To test whether Aβ₁₋₄₂ specifically inhibits nAChRs, the inventors also examined the effects of 1 nM Aβ₁₋₄₂ on GABA- or glutamate-induced currents in rat MS/DB cholinergic neurons, and the results demonstrated that both GABA_{A} receptors and ionotropic glutamate receptors were insensitive to inhibition by 1 nM Aβ₁₋₄₂ even when peptide effects on ACh-induced current were evident (FIG. 10). Collectively, these results indicate that, under our experimental conditions, pathologically-relevant, low nM concentrations of Aβ₁₋₄₂, especially in an oligomeric form, specifically inhibit function of apparently heteromeric α7β2-nAChRs, but peptides cannot inhibit function of homomeric α7-nAChRs, GABA_{A}, or glutamate receptors on MS/DB cholinergic neurons.

### Example 15

### Heteromeric α7β2-nAChRs Heterologously Expressed in Xenopus Oocytes Display Slower Current Kinetics and High Sensitivity to Aβ₁₋₄₂

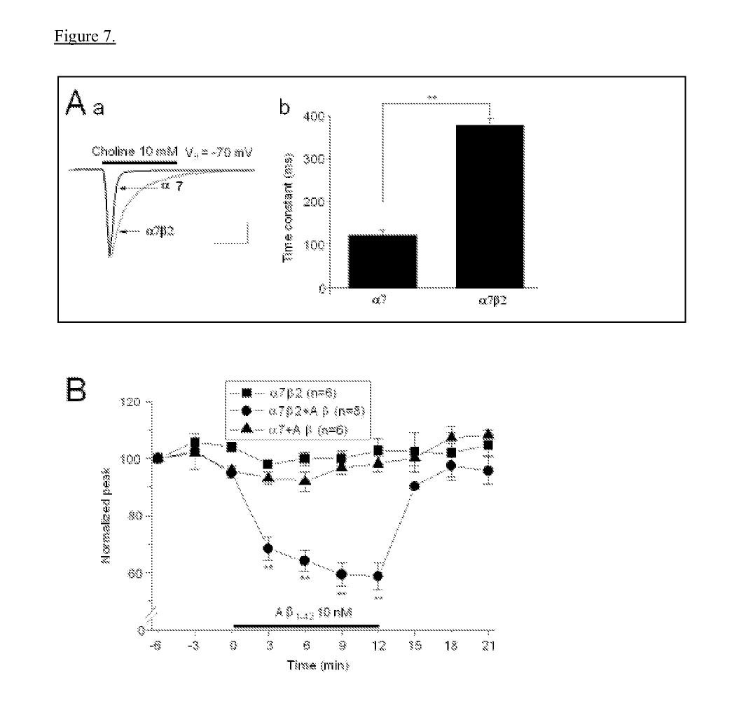

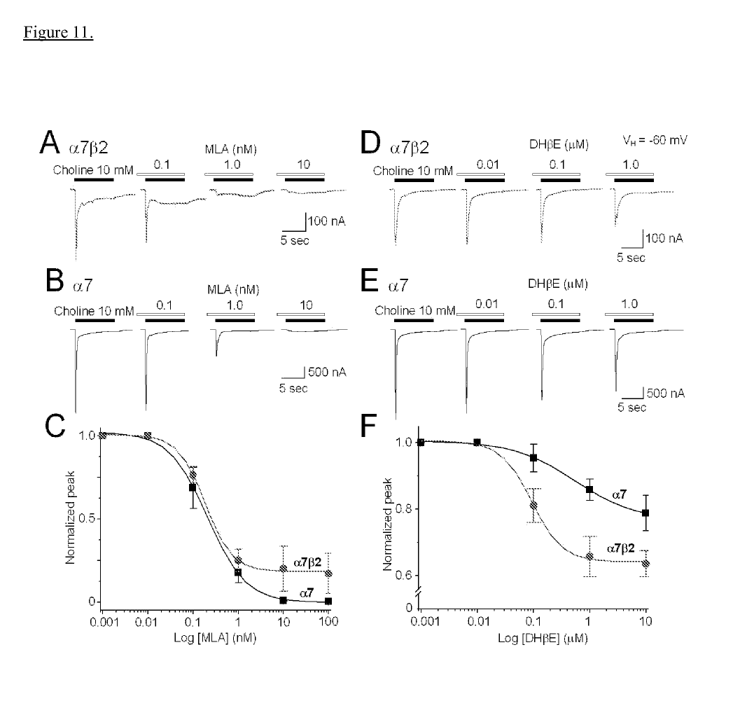

To further investigate features of presumed, novel α7β2-nAChRs as naturally expressed in basal forebrain cholinergic neurons, the inventors introduced nAChR α7 subunits alone or in combination with β2 subunits into Xenopus oocytes. Compared to homomeric α7-nAChRs (FIG. 7Aa), heteromeric α7β2-nAChRs expressed in oocytes injected with rat nAChR α7 and β2 subunit cRNAs at a ratio of 1:1 exhibited smaller peak current responses to choline and slower current decay rates (FIG. 7Ab). These results are consistent with findings in a previous report (Khiroug et al., 2002). As was the case for comparisons between native nAChR responses in rat MS/DB or VTA neurons (FIG. 4), sensitivity to functional blockade by MLA was similar for heterologously expressed α7β2- or α7-nAChR (FIG. 11A-C). Also similar to the case for native nAChR, heterologously expressed α7β2-nAChR were more sensitive to blockade by DHβE than were homomeric α7-nAChR. (Wang et al., 2000) indicates presence of β2 subunits with α7 subunits in rodent MS/DB neurons. The inventors then tested the sensitivity of heterologously-expressed α7β2-nAChRs in oocytes to Aβ. As was the case for presumed, native α7β2-nAChRs on MS/DB neurons, heterologously-expressed heteromeric α7β2-nAChRs, but not homomeric α7-nAChRs, demonstrated sensitivity to Aβ₁₋₄₂ (10 nM) and insensitivity to 10 nM scrambled Aβ₁₋₄₂ (FIG. 7B). These results obtained using heterologously-expressed nAChRs again are consistent with the hypothesis that nAChR α7 and β2 subunits likely co-assemble and form a unique α7β2-nAChR that enhances receptor sensitivity to pathologically-relevant, low nM concentrations of Aβ₁₋₄₂.

### Example 16

### Basal Forebrain nAChRs in nAChR β2 Subunit-Null Mice do not Show Co-Immunoprecipitation of nAChR α7 and β2 Subunits, Exhibit Fast Whole-Cell Current Kinetics, and Show Low Sensitivity to Aβ₁₋₄₂

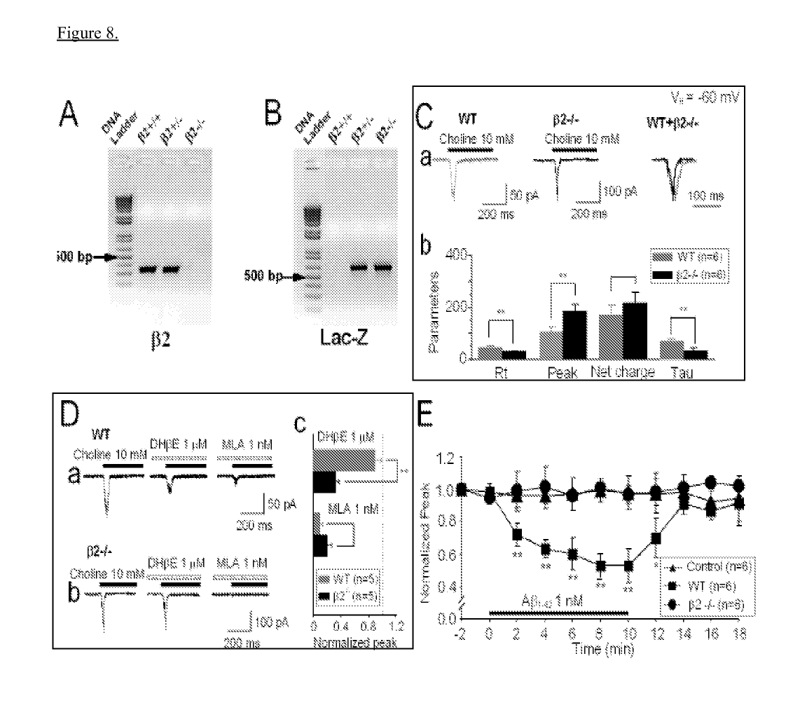

As further support for the concept that basal forebrain cholinergic neurons express novel α7β2-nAChRs, the inventors used wild-type and nAChR β2 subunit knockout (β2^{-/-}) mice. PCR genotyping was used to identify wild-type or β2^{-/-} mice (FIG. 8A, B). Using the immunoprecipitation protocol previously described and protein extracts from the MS/DB, nAChR β2 subunits were found to co-precipitate with nAChR α7 subunits only for samples from wild-type but not from β2 mice (FIG. 3E, right panels). Choline-induced currents in MS/DB cholinergic neurons dissociated from β2^{-/-} mice exhibited higher current amplitude, faster kinetics (FIG. 8C), and lower sensitivity to DHβE (FIG. 8Da-c) than responses in cholinergic neurons dissociated from wild-type mice. As expected, 1 nM Aβ₁₋₄₂ failed to suppress choline-induced currents in MS/DB neurons from β2^{-/-} mice but did suppress choline-induced currents in MS/DB neurons from wild-type mice (FIG. 8E). These results again strongly support the concept that heteromeric, functional α7β2-nAChRs on basal forebrain MS/DB cholinergic neurons are highly sensitive to a pathologically-relevant concentrations of Aβ₁₋₄₂.

### Example 17

### Novel, Heteromeric, Functional α7β2-nAChR Subtype

nAChRs in basal forebrain participate in cholinergic transmission and cognitive processes associated with learning and memory (Levin and Rezvani, 2002; Mansvelder et al., 2006). During the early stages of AD, decreases in nAChR-like radioligand binding sites have been observed (Burghaus et al., 2000; Nordberg, 2001), suggesting that nAChR dysfunction could be involved in AD pathogenesis and cholinergic deficiencies (Nordberg, 2001). Evidence indicates that enhancement of α7-nAChR function protects neurons against A [symbol] toxicity through any or some combination of a number of different mechanisms, as outlined previously (Sadot et al., 1996; Lahiri et al., 2002; Nagele et al., 2002; Geerts, 2005; Liu et al., 2007a). On the other hand, pharmacological interventions or diminished nAChR expression produces learning and memory deficits (Levin and Rezvani, 2002).

Findings described herein are consistent with the natural expression of a novel, heteromeric, functional α7β2-nAChR subtype on forebrain cholinergic neurons that is particularly sensitive to functional inhibition by a pathologically-relevant concentration (1 nM) of Aβ₁₋₄₂. Some previous studies investigating the acute effects of Aβ₁₋₄₂ on nAChRs examined receptors on neurons from regions other than the basal forebrain or that were heterologously expressed (Liu et al., 2001; Pettit et al., 2001; Grassi et al., 2003; Wu et al., 2004a; Lamb et al., 2005; Pym et al., 2005) and/or used Aβ peptides at concentrations (between 100 nM and 10 μM) that greatly exceed Aβ concentrations found in AD brain (Kuo et al., 2000; Mehta et al., 2000). Other studies identified α7-nAChR-like, ACh-induced currents in MS/DB cholinergic neurons using slice-patch recordings (Henderson et al., 2005; Thinschmidt et al., 2005) and characterized functional, non-α7-nAChRs using acutely-dissociated forebrain neurons (Fu and Jhamandas, 2003). Studies described herein combined whole-cell current recordings from acutely-dissociated neurons and investigation of MS/DB cholinergic neuronal nAChRs to identify functional nAChRs that have some features of receptors containing α7 subunits, but also found high sensitivity of these nAChRs to low concentrations of Aβ₁₋₄₂. Studies described herein are consistent with other previous findings and also indicate that functional α7β2-nAChRs can be heterologously expressed in oocytes. Histological studies have demonstrated co-expression of nAChR α7 and β2 subunits in most forebrain cholinergic neurons (Azam et al., 2003). The results also are consistent with those observations and show cell-specific, co-expression of nAChR α7 and β2 subunits at both message and protein levels. There are other reports (Yu and Role, 1998); (El-Hajj et al., 2007) that nAChR α7 subunits could be co-assembled with other subunits to form native, heteromeric, α7*-nAChRs. These findings herein are consistent with those observations. The notion that the Aβ₁₋₄₂-sensitive, functional nAChR subtype in MS/DB neurons displaying some features of nAChRs containing α7 subunits, but distinctive from homomeric α7-nAChRs, is composed of α7 and β2 subunits, is supported by the loss of Aβ sensitivity and the conversion of functional nAChR properties to those like homomeric α7-nAChRs in nAChR β2 subunit knockout animals. It has been reported that there are two isoforms (α7-1 and α7-2) of α7-nAChR transcript in homomeric α7-nAChRs. The α7-2 transcript that contains a novel exon is widely expressed in the brain and showed very slow current kinetics (Severance et al., 2004); (Severance and Cuevas, 2004); (Saragoza et al., 2003). However, the inventors contend that the heteromeric α7β2-nAChR described in the present study and expressed in MS/DB neurons is not a homomeric nAChR composed of or containing the α7-2 transcript for three reasons: (1) in β2^{-/-} mice, α7-nAChR-like whole-cell current responses to choline acquire fast kinetic characteristics like those of α7-nAChR responses in VTA neurons, (2) immunoprecipitation-western blot analyses show co-assembly of α7 and β2 subunits from the MS/DB but not from the VTA, nor from the MS/DB of β2^{-/-} mice, and (3) pharmacologically heteromeric α7β2-nAChRs were sensitive not only to MLA, but also to DHβE.

A recent study suggested that levels of oligomeric forms of Aβ₁₋₄₂, rather than monomers or Aβ fibrils, most closely correlate with cognitive dysfunction in animal models of AD (Haass and Selkoe, 2007). The inventors' findings also convey that Aβ oligomers have the most profound effects on nAChR function, thus extending earlier studies of Aβ-nAChR interactions (Wu et al., 2004a) and illuminating why there have been apparent discrepancies in some of the earlier work concerning Aβ-nAChR interactions.

Alzheimer's disease (AD) is a dementing, neurodegenerative disorder characterized by accumulation of amyloid β (Aβ) peptide-containing neuritic plaques, degeneration of basal forebrain cholinergic neurons, and gradually impaired learning and memory (Selkoe, 1999). The extent of learning and memory deficits in AD is proportional to the degree of forebrain cholinergic neuronal degeneration, and the extent of Aβ deposition is used to characterize disease severity (Selkoe, 1999). Processes such as impaititient of neurotrophic support and disorders in glucose metabolism have been implicated in cholinergic neuronal loss and AD (Dolezal and Kasparova, 2003). However, clear neurotoxic effects of Aβ across a range of in vivo and in vitro models suggest that Aβ plays potentially causal roles in cholinergic neuronal degeneration and consequent learning and memory deficits (Selkoe, 1999).

Based on the findings described herein, selective, high-affinity effects of oligomeric Aβ₁₋₄₂ on basal forebrain, cholinergic neuronal α7β2-nAChRs acutely contribute to disruption of cholinergic signaling and diminished learning and memory abilities (Yan and Feng, 2004). Moreover, to the extent that basal forebrain cholinergic neuronal health requires activity of α7β2-nAChRs, inhibition of α7β2-nAChR function by oligomeric Aβ₁₋₄₂ can lead to losses of trophic support for those neurons and/or their targets, and cross-catalyzed spirals of receptor functional loss and neuronal degeneration also can contribute to the progression of AD. Drugs targeting α7β2-nAChRs to protect them against Aβ effects or restoration of α7β2-nAChR function in cholinergic forebrain neurons will serve as viable therapies for AD.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

Alkondon, M., (2008) A single in vivo application of cholinesterase inhibitors has neuron type-specific effects on nicotinic receptor activity in guinea pig hippocampus. Journal of Pharmacology and Experimental Therapeutics 328: 69-82, 2009.
Azam L, Winzer-Serhan U, Leslie F M (2003) Co-expression of alpha7 and beta2 nicotinic acetylcholine receptor subunit mRNAs within rat brain cholinergic neurons. Neuroscience 119:965-977.
Burghaus L, Schutz U, Krempel U, de Vos R A, Jansen Steur E N, Wevers A, Lindstrom J, Schroder H (2000) Quantitative assessment of nicotinic acetylcholine receptor proteins in the cerebral cortex of Alzheimer patients. Brain Res Mol Brain Res 76:385-388.
Counts S E, He B, Che 5, lkonomovic MD, DeKosky S T, Ginsberg S D, Mufson E J (2007) Alpha7 nicotinic receptor up-regulation in cholinergic basal forebrain neurons in Alzheimer disease. Arch Neurol 64:1771-1776.
Dineley K T, Bell K A, Bui D, Sweatt J D (2002a) beta-Amylaid peptide activates alpha 7 nicotinic acetylcholine receptors expressed in Xenopus oocytes. J Biol Chem 277:25056-25061.
Dineley K T, Xia X, Bui D, Sweatt J D, Zheng H (2002b) Accelerated plaque accumulation, associative learning deficits, and up-regulation of alpha 7 nicotinic receptor protein in transgenic mice co-expressing mutant human presenilin 1 and amyloid precursor proteins. J Biol Chem 277:22768-22780.
Dolezal V, Kasparova J (2003) Beta-amyloid and cholinergic neurons. Neurochem Res 28:499-506.
Dougherty J J, Wu J, Nichols R A (2003) Beta-amyloid regulation of presynaptic nicotinic receptors in rat hippocampus and neocortex. J Neurosci 23:6740-6747.
El-Hajj R A, McKay S B, McKay D B (2007) Pharmacological and immunological identification of native alpha7 nicotinic receptors: evidence for homomeric and heteromeric alpha7 receptors. Life Sci 81:1317-1322.
Flores C M, Rogers S W, Pabreza L A, Wolfe B B, Kellar K J (1992) A subtype of nicotinic cholinergic receptor in rat brain is composed of alpha 4 and beta 2 subunits and is up-regulated by chronic nicotine treatment. Mol Pharmacol 41:31-37.
Fu W, Jhamandas J H (2003) Beta-amyloid peptide activates non-alpha7 nicotinic acetylcholine receptors in rat basal forebrain neurons. J Neurophysiol 90:3130-3136.
Geerts H (2005) Indicators of neuroprotection with galantamine. Brain Res Bull 64:519-524.
Gopalakrishnan M, Monteggia L M, Anderson D J, Molinari E J, Piattoni-Kaplan M, Donnelly-Roberts D, Arneric S P, Sullivan J P (1996) Stable expression, pharmacologic properties and regulation of the human neuronal nicotinic acetylcholine alpha 4 beta 2 receptor. J Pharmacol Exp Ther 276:289-297.
Grassi F, Palma E, Tonini R, Amici M, Ballivet M, Eusebi F (2003) Amyloid beta(1-42) peptide alters the gating of human and mouse alpha-bungarotoxin-sensitive nicotinic receptors. J Physiol 547:147-157.
Haass C, Selkoe D J (2007) Soluble protein oligomers in neurodegeneration: lessons from the Alzheimer's amyloid beta-peptide. Nat Rev Mol Cell Biol 8:101-112.
Henderson Z, Boros A, Janzso G, Westwood A J, Monyer H, Halasy K (2005) Somato-dendritic nicotinic receptor responses recorded in vitro from the medial septal diagonal band complex of the rodent. J Physiol 562:165-182.
Horn R, Marty A (1988) Muscarinic activation of ionic currents measured by a new whole-cell recording method. J Gen Physiol 92:145-159.
Jensen A A, Frolund B, Liljefors T, Krogsgaard-Larsen P (2005) Neuronal nicotinic acetylcholine receptors: structural revelations, target identifications, and therapeutic inspirations. J Med Chem 48:4705-4745.
Khiroug S S, Harkness P C, Lamb P W, Sudweeks S N, Khiroug L, Millar N S, Yakel J L (2002) Rat nicotinic ACh receptor alpha7 and beta2 subunits co-assemble to form functional heteromeric nicotinic receptor channels. J Physiol 540:425-434.
Kuo Y M, Kokjohn T A, Watson M D, Woods A S, Cotter R J, Sue L I, Kalback W M, Emmerling M R, Beach T G, Roher A E (2000) Elevated abeta42 in skeletal muscle of Alzheimer disease patients suggests peripheral alterations of AbetaPP metabolism. Am J Pathol 156:797-805.
Lahiri D K, Utsuki T, Chen D, Farlow M R, Shoaib M, Ingram D K, Greig N H (2002) Nicotine reduces the secretion of Alzheimer's beta-amyloid precursor protein containing beta-amyloid peptide in the rat without altering synaptic proteins. Ann N Y Acad Sci 965:364 372.
Lamb P W, Melton M A, Yakel J L (2005) Inhibition of neuronal nicotinic acetylcholine receptor channels expressed in Xenopus oocytes by beta-amyloid1-42 peptide. J Mol Neurosci 27:13-21.
Levin E D, Rezvani A H (2002) Nicotinic treatment for cognitive dysfunction. Curr Drug Targets CNS Neurol Disord 1:423-431.
Lindstrom J (1996) Neuronal nicotinic acetylcholine receptors. Ion Channels 4:377-450.
Lindstrom J, Anand R, Gerzanich V, Peng X, Wang F, Wells G (1996) Structure and function of neuronal nicotinic acetylcholine receptors. Prog Brain Res 109:125-137.
Liu Q, Kawai H, Berg D K (2001) beta-Amyloid peptide blocks the response of alpha 7-containing nicotinic receptors on hippocampal neurons. Proc Natl Acad Sci USA 98:4734-4739.
Liu Q, Zhang J, Zhu H, Qin C, Chen Q, Zhao B (2007a) Dissecting the signaling pathway of nicotine-mediated neuroprotection in a mouse Alzheimer disease model. FASEB J 21:61-73.
Liu Z, Zhang J, Berg D K (2007b) Role of endogenous nicotinic signaling in guiding neuronal development. Biochem Pharmacol 74:1112-1119.
   Lukas R J, Changeux J P, Le Novere N, Albuquerque E X, Balfour D J, Berg D K, Bertrand D, Chiappinelli V A, Clarke P B, Collins A C, Dani J A, Grady S R, Kellar K J, Lindstrom J M, Marks M J, Quik M, Taylor P W, Wonnacott S (1999) International Union of Pharmacology. XX. Current status of the nomenclature for nicotinic acetylcholine receptors and their subunits. Pharmacol Rev 51:397-401.
Mansvelder H D, van Aerde K I, Couey J J, Brussaard A B (2006) Nicotinic modulation of neuronal networks: from receptors to cognition. Psychopharmacology (Bed) 184:292-305.
Mehta P D, Pirttila T, Mehta S P, Sersen E A, Aisen P S, Wisniewski H M (2000) Plasma and cerebrospinal fluid levels of amyloid beta proteins 1-40 and 1-42 in Alzheimer disease. Arch Neurol 57:100-105.
Moser N, Mechawar N, Jones I, Gochberg-Sarver A, Orr-Urtreger A, Plomann M, Sales R, Mattes B, Marubio L, Roth U, Maskos U, Winzer-Serhan U, Bourgeois J P, Le Sourd A M, De Blasi M, Schroder H, Lindstrom J, Maelicke A, Changeux J P, Wevers A (2007) Evaluating the suitability of nicotinic acetylcholine receptor antibodies for standard immunodetection procedures. J Neurochem 102:479-492.
Mudo G, Belluardo N, Fuxe K (2007) Nicotinic receptor agonists as neuroprotective/neurotrophic drugs. Progress in molecular mechanisms. J Neural Transm 114:135-147.
Nagele R G, D'Andrea M R, Anderson W J, Wang H Y (2002) Intracellular accumulation of beta-amyloid(1-42) in neurons is facilitated by the alpha 7 nicotinic acetylcholine receptor in Alzheimer's disease. Neuroscience 110:199-211.
Nordberg A (2001) Nicotinic receptor abnormalities of Alzheimer's disease: therapeutic implications. Biol Psychiatry 49:200-210.
Pettit D L, Shao Z, Yakel J L (2001) beta-Amyloid(1-42) peptide directly modulates nicotinic receptors in the rat hippocampal slice. J Neurosci 21:RC120.
Pym L, Kemp M, Raymond-Delpech V, Buckingham S, Boyd C A, Sattelle D (2005) Subtype-specific actions of beta-amyloid peptides on recombinant human neuronal nicotinic acetylcholine receptors (alpha7, alpha4beta2, alpha3beta4) expressed in Xenopus laevis oocytes. Br J Pharmacol 146:964-971.
Sadot E, Gurwitz D, Barg J, Behar L, Ginzburg I, Fisher A (1996) Activation of m1 muscarinic acetylcholine receptor regulates tau phosphorylation in transfected PC12 cells. J Neurochem 66:877-880.
Saragoza P A, Modir J G, Goel N, French K L, Li L, Nowak M W, Stitzel J A (2003) Identification of an alternatively processed nicotinic receptor alpha7 subunit RNA in mouse brain. Brain Res Mol Brain Res 117:15-26.
Selkoe D J (1999) Translating cell biology into therapeutic advances in Alzheimer's disease. Nature 399:A23-31.
Severance E G, Cuevas J (2004) Distribution and synaptic localization of nicotinic acetylcholine receptors containing a novel alpha7 subunit isoform in embryonic rat cortical neurons. Neurosci Lett 372:104-109.
Severance E G, Zhang H, Cruz Y, Pakhlevaniants S, Hadley S H, Amin J, Wecker L, Reed C, Cuevas J (2004) The alpha7 nicotinic acetylcholine receptor subunit exists in two isoforms that contribute to functional ligand-gated ion channels. Mol Pharmacol 66:420-429.
Sivaprakasam K (2006) Towards a unifying hypothesis of Alzheimer's disease: cholinergic system linked to plaques, tangles and neuroinflammation. Curr Med Chem 13:2179-2188.
Thinschmidt J S, Frazier C J, King M A, Meyer E M, Papke R L (2005) Medial septal/diagonal band cells express multiple functional nicotinic receptor subtypes that are correlated with firing frequency. Neurosci Lett 389:163-168.
Wang H Y, Lee D H, D'Andrea M R, Peterson P A, Shank R P, Reitz A B (2000) beta-Amyloid(1-42) binds to alpha7 nicotinic acetylcholine receptor with high affinity. Implications for) Alzheimer's disease pathology. J Biol Chem 275:5626-5632.
Whiting P J, Schoepfer R, Swanson L W, Simmons D M, Lindstrom J M (1987) Functional acetylcholine receptor in PC12 cells reacts with a monoclonal antibody to brain nicotinic receptors. Nature 327:515-518.
Wu J, Chan P, Schroeder K M, Ellsworth K, Partridge L D (2002) 1-Methyl-4-phenylpridinium (MPP+)-induced functional run-down of GABA(A) receptor-mediated currents in acutely dissociated dopaminergic neurons. J Neurochem 83:87-99.
Wu J, Kuo Y P, George A A, Xu L, Hu J, Lukas R J (2004a) beta-Amyloid directly inhibits human alpha4beta2-nicotinic acetylcholine receptors heterologously expressed in human SH-EP1 cells. J Biol Chem 279:37842-37851.)
Wu J, George A A, Schroeder K M, Xu L, Marxer-Miller S, Lucero L, Lukas R J (2004b) Electrophysiological, pharmacological, and molecular evidence for alpha7-nicotinic acetylcholine receptors in rat midbrain dopamine neurons. J Pharmacol Exp Ther 311:80-91.
Yan Z, Feng J (2004) Alzheimer's disease: interactions between cholinergic functions and beta-amyloid. Curr Alzheimer Res 1:241-248.
Yu C R, Role L W (1998) Functional contribution of the alpha5 subunit to neuronal nicotinic channels expressed by chick sympathetic ganglion neurones. J Physiol 509 (Pt 3):667-681.

1. A method of treating a neurodegenerative disorder in an individual, comprising:
providing a composition capable of inhibiting dysfunctional signaling of α7 nicotinic acetylcholine receptors (nAChRs); and
administering a therapeutically effective amount of the composition to inhibit dysfunctional signaling of α7 nAChRs to treat the neurodegenerative disorder.

2. The method of claim 1, wherein the α7 nAChRs comprise heteromeric α7β2 nAChRs.

3. The method of claim 1, wherein the composition capable of inhibiting dysfunctional signaling of α7 nAChRs comprises a β2 nAChR antagonist.

4. The method of claim 1, wherein the neurodegenerative disorder comprises Alzheimer's Disease, dementia and/or epilepsy.

5. The method of claim 1, wherein the neurodegenerative disorder comprises an early stage form of Alzheimer's Disease.

6. The method of claim 1, wherein the composition capable of inhibiting dysfunctional signaling of α7 nAChRs comprises an α7 nAChR antagonist.

7. The method of claim 1, wherein the composition capable of inhibiting dysfunctional signaling of α7 nAChRs comprises a compound comprising kynurenic acid (KYNA), methyllycaconitine (MLA), α-bungarotoxin (BGT), cholinesterase inhibitor, memantine, and/or α-conotoxin, or a pharmaceutical equivalent, derivative, analog and/or salt thereof.

8. The method of claim 1, wherein inhibiting the dysfunctional signaling of α7 nAChRs comprises restoring function of heteromeric α7β2 nAChRs.

9. The method of claim 1, wherein inhibiting the dysfunctional signaling of α7 nAChRs comprises protecting heteromeric α7β2 nAChRs from amyloid β (Aβ) effects.

10. The method of claim 1, wherein the individual is a human.

11. The method of claim 1, wherein the individual is a rodent.

12. The method of claim 1, wherein the dysfunctional signaling of α7 nAChRs occurs in the brain medial septum and/or diagonal band in the individual.

13. A method of diagnosing a neurodegenerative disorder in an individual, comprising:
obtaining a sample from the individual;
assaying the sample to determine the presence or absence of dysfunctional signaling of α7 nicotinic acetylcholine receptors (nAChRs) in the individual; and
diagnosing the neurodegenerative disorder based on the presence of dysfunctional signaling of α7 nAChRs in the individual.

14. The method of claim 13, wherein the α7 nAChRs comprise heteromeric α7β2 nAChRs.

15. The method of claim 13 wherein the individual is a human.

16. The method of claim 13 wherein the individual is a rodent.

17. The method of claim 13 wherein the neurodegenerative disorder comprises Alzheimer's Disease, dementia and/or epilepsy.

18. The method of claim 13 wherein the dysfunctional signaling of α7 nAChRs occurs in the brain medial septum and/or diagonal band in the individual.

19. The method of claim 13 wherein the neurodegenerative disorder is non-responsive to treatment with galantamine, or a pharmaceutical equivalent, derivative, analog and/or salt thereof.

20. The method of claim 13, wherein prior to obtaining the sample the individual is suspected of having a neurodegenerative disorder.

21. A method of prognosing the onset of Alzheimer's Disease and/or dementia in an individual, comprising:
obtaining a sample from the individual;
assaying the sample to determine the presence or absence of dysfunctional signaling of α7 nicotinic acetylcholine receptors (nAChRs) in the individual; and
prognosing the onset of Alzheimer's Disease and/or dementia based on the presence of dysfunctional signaling of α7 nAChRs in the individual.

22. The method of claim 21 herein the α7 nAChRs comprise heteromeric α7β2 nAChRs.

23. The method of claim 21 wherein the dysfunctional signaling of α7 nAChRs occurs in the brain medial septum and/or diagonal band in the individual.

24. A method of diagnosing an increased likelihood of an individual developing a neurodegenerative disorder relative to a normal subject, comprising:
obtaining a sample from the individual;
assaying the sample to determine the presence or absence of dysfunctional signaling of α7 nicotinic acetylcholine receptors (nAChRs) in the individual; and
diagnosing an increased likelihood of developing the neurodegenerative disorder relative to the normal subject based on the presence of dysfunctional signaling of α7 nAChRs in the individual.

25. The method of claim 24, wherein the α7 nAChRs comprise heteromeric α7β2 nAChRs.

26. The method of claim 24, wherein the neurodegenerative disorder comprises Alzheimer's Disease, dementia and/or epilepsy.

27. The method of claim 24, wherein prior to obtaining the sample the individual is suspected of having a neurodegenerative disorder.


SEQUENCE LISTING (8 sequences)

SEQ ID NO: 1 - DNA, 45 bp, Homo sapiens
  misc_feature (22)..(22): n is a, c, g, or t
atttaggtgacactatagaagnggatcatcgtgggcctctcagtg

SEQ ID NO: 2 - DNA, 44 bp, Homo sapiens
taatacgactcactatagggagagttggcgatgtagcggacctc

SEQ ID NO: 3 - DNA, 47 bp, Homo sapiens
  misc_feature (22)..(22): n is a, c, g, or t
atttaggtgacactatagaagngtcacggtgttcctgctgctcatct

SEQ ID NO: 4 - DNA, 47 bp, Homo sapiens
taatacgactcactatagggagatcctccctcacactctggtcatca

SEQ ID NO: 5 - DNA, 32 bp, Homo sapiens
cggagcatttgaactctgagcagtggggtcgc

SEQ ID NO: 6 - DNA, 24 bp, Homo sapiens
ctcgctgacacaagggctgcggac

SEQ ID NO: 7 - DNA, 26 bp, Homo sapiens
cactacgtctgaacgtcgaaaacccg

SEQ ID NO: 8 - DNA, 26 bp, Homo sapiens
cgggcaaataatatcggtggccgtgg